(19) United States
(12) Patent Application Publication
(10) Pub. No.: US 2023/0122920 A1
(43) Pub. Date: Apr. 20, 2023

(54) PATHWAY GENERATION APPARATUS, PATHWAY GENERATION METHOD, AND PATHWAY GENERATION PROGRAM

(71) Applicant: FRONTEO, Inc., Tokyo (JP)

(72) Inventor: Hiroyoshi TOYOSHIBA, Tokyo (JP)

(21) Appl. No.: 17/995,338

(22) PCT Filed: Nov. 26, 2020
(86) PCT No.: PCT/JP2020/043961

§ 371 Date: Oct. 3, 2022

(30) Foreign Application Priority Data
Jul. 2, 2020 (JP) 2020-114658

(51) Int. Cl. G16H 70/60

(52) U.S. Cl. CPC G16H 70/60

(57) ABSTRACT

Included are a related molecule estimation unit 12 that inputs a disease feature vector specified for a disease to be analyzed to a first trained model, thereby estimating a plurality of molecules related to the disease, a molecular property estimation unit 13 that inputs a disease feature vector and a molecule feature vector specified for the plurality of molecules estimated by the related molecule estimation unit 12 to a second trained model, thereby estimating a probability that a property of a molecule acting on the disease is causative, and a pathway generation unit 14 that generates a pathway representing an intermolecular interaction as a route map in a manner that a causative molecule is on an upstream side and a responsive molecule is on an downstream side and that a known intermolecular connection relationship is reflected by using a property of a molecule estimated by the molecular property estimation unit 13.

### TECHNICAL FIELD

The present invention relates to a pathway generation apparatus, a pathway generation method, and a pathway generation program, and is particularly suitable for use in a technology for generating a pathway representing an intermolecular interaction as a route map.

### BACKGROUND ART

Conventionally, there has been a known pathway representing an intermolecular interaction as a route map. The pathway represents a molecule of a gene, a protein, etc. using a symbol such as a circle or a square, and is expressed by connecting symbols with arrows that represent intermolecular interactions. Such visualization of the intermolecular interaction allows easier understanding of life phenomena such that it is possible to investigate a path containing a gene group whose expression level has changed. For example, a pathway is widely used in a field of disease treatment or drug discovery.

There are two types of pathways, one created manually and the other created using a computer. The former pathway is created mainly by researchers reading biochemical or medical literature and drawing content described therein as a text as a route map of the pathway. The latter pathway is created, for example, by reading a text described in a literature as text data and depicting described content whose meaning is interpreted by natural language processing as a route map.

However, a conventional pathway created manually is merely obtained by a creator depicting a known intermolecular interaction understood from description of a literature as a pathway. Therefore, the pathway that can be manually created is limited to a range of described content of the literature read by the creator. A conventional pathway created by a computer is also basically similar thereto, and the pathway that can be created is limited to a range of described content of a literature read by the computer as text data. More literature can be read in the case of a computer than in the case of a human, and a width of a pathway that can be created increases accordingly. However, a known intermolecular interaction described in the literature is merely depicted.

Note that there has been a known method of predicting a protein-protein interaction having potential as a drug target by performing supervised machine learning with predetermined attributes related to proteins as feature vectors (for example, see Patent Document 1). In a prediction system described in Patent Document 1, machine learning is performed using an attribute of a biological function of each protein as one predetermined attribute related to the protein. Patent Document 1 discloses that the number of pathways containing each protein is used as one attribute of the biological function of each protein.

However, a technology described in Patent Document 1 does not create a pathway using machine learning, and performs machine learning using attributes related to a plurality of pathways that have been previously created, and Patent Document 1 fails to disclose a method of creating a pathway.

Patent Document 1: JP-A-2010-165230

### SUMMARY OF THE INVENTION

### Technical Problem

A pathway created by a conventional method can be used when researching and developing a new drug or a new treatment effective against a disease for which an effective treatment or drug has not been established, etc. However, since the conventional pathway merely depicts the known intermolecular interaction described in the literature, etc., it is difficult to obtain knowledge beyond human intelligence from the pathway. In particular, for a newly developed disease of an unknown property or a pathogen of unknown identity, etc., there is a problem that it is difficult to obtain, from the conventional pathway, knowledge such as a type of molecule involved or a type of existing drug which is effective as a research target.

The invention has been made in view of such circumstances, and an object of the invention is to allow generation of a pathway useful for obtaining new knowledge beyond a range of a known intermolecular interaction described in a literature, etc.

### Solution to Problem

To solve the above-mentioned problem, the invention includes a related molecule estimation unit that inputs a disease feature vector specified for a disease to be analyzed to a first trained model, thereby estimating a plurality of molecules related to the disease, a molecular property estimation unit that inputs a disease feature vector specified for the disease to be analyzed and a molecule feature vector specified for the plurality of molecules estimated by the related molecule estimation unit to a second trained model, thereby estimating a probability that a molecule is causative or responsive as a property acting on the disease for each of the plurality of molecules, and a pathway generation unit that generates a pathway representing an intermolecular interaction as a route map in a manner that a causative molecule is on an upstream side and a responsive molecule is on an downstream side and that an intermolecular connection relationship shown by a known knowledge database is reflected for the plurality of molecules estimated by the related molecule estimation unit by using a property of a molecule estimated by the molecular property estimation unit and the knowledge database showing the intermolecular connection relationship.

### Advantageous Effects of the Invention

According to the invention configured as described above, when a disease feature vector for a disease to be analyzed is input to a first trained model, not only a molecule known to be related to the disease, but also a molecule whose relevance to the disease is unknown may be output as a related molecule by estimation based on learning. In addition, when a molecule feature vector of the molecule estimated in this way and the disease feature vector are input to a second trained model, a probability indicating whether a molecule may exhibit a causative or responsive property with respect to the disease is output not only for the molecule whose relevance to the disease is known but also the molecule whose relevance is unknown by estimation based on learning. Then, an estimation result with regard to a property of a molecule presumed to be associated with the disease in this way and a known knowledge database showing an intermolecular connection relationship are used to generate a pathway representing an intermolecular interaction as a route map. In this way, it is possible to generate a pathway useful for obtaining new knowledge beyond a range of a known intermolecular interaction described in a literature, etc.

### BRIEF DESCRIPTION OF THE DRAWINGS

FIG. 1 is a diagram illustrating an overall configuration example of a pathway providing system including a pathway generation apparatus according to the present embodiment.

FIG. 2 is a block diagram illustrating a functional configuration example of a server apparatus (pathway generation apparatus) according to the present embodiment.

FIG. 3 is a block diagram illustrating a functional configuration example of a client terminal according to the present embodiment.

FIG. 4 is a block diagram illustrating a functional configuration example of a feature vector computation apparatus.

FIG. 5 is a diagram illustrating an example of a disease feature vector and a molecule feature vector.

FIG. 6 is a diagram illustrating an example of a pathway displayed on a display apparatus.

FIG. 7 is a flowchart illustrating an operation example of a server apparatus (pathway generation apparatus) according to the present embodiment.

### MODE FOR CARRYING OUT THE INVENTION

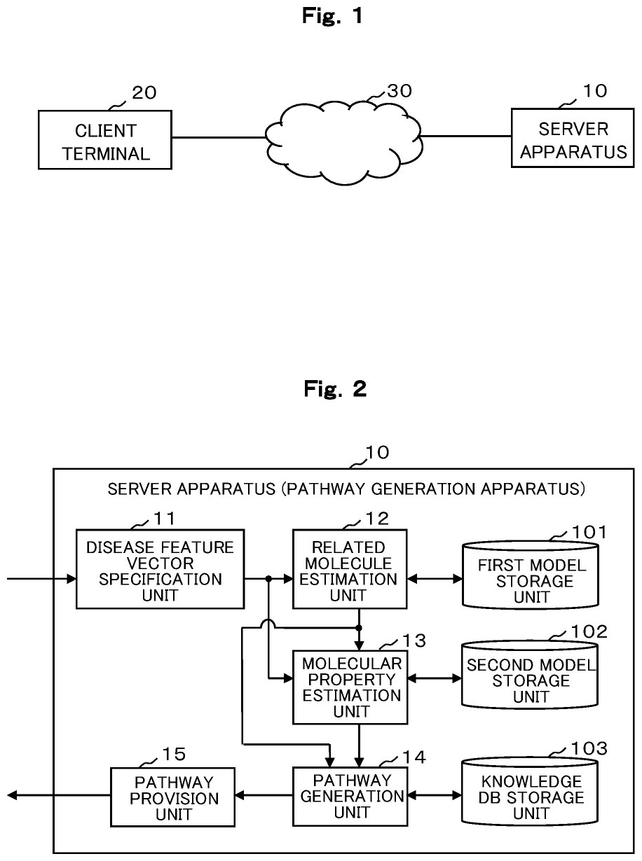

Hereinafter, an embodiment of the invention will be described with reference to the drawings. FIG. 1 is a diagram illustrating an overall configuration example of a pathway providing system including a pathway generation apparatus according to the present embodiment. As illustrated in FIG. 1, the pathway providing system of the present embodiment includes a server apparatus 10 and a client terminal 20, and the server apparatus 10 and the client terminal 20 are connected by a communication network 30 such as the Internet. The server apparatus 10 includes the pathway generation apparatus of the present embodiment.

In the pathway providing system of the present embodiment, when a disease is designated from the client terminal 20 and the server apparatus 10 is requested to provide a pathway, the server apparatus 10 generates a pathway representing as a route map an intermolecular interaction related to the designated disease, and provides the generated pathway to the client terminal 20. The client terminal 20 displays the pathway provided from the server apparatus 10 on the display apparatus. The client terminal 20 can perform such a process using a web browser, for example.

FIG. 2 is a block diagram illustrating a functional configuration example of the server apparatus 10 (pathway generation apparatus) according to the present embodiment. As illustrated in FIG. 2, the server apparatus 10 of the present embodiment includes a disease feature vector specification unit 11, a related molecule estimation unit 12, a molecular property estimation unit 13, a pathway generation unit 14, and a pathway provision unit 15 as functional configurations. In addition, the server apparatus 10 of the present embodiment includes a first model storage unit 101, a second model storage unit 102, and a knowledge DB storage unit 103 as storage media. The pathway generation apparatus of the present embodiment includes blocks other than the pathway provision unit 15. Note that the disease feature vector specification unit 11 may be provided in the client terminal 20.

Each of the above functional blocks 11 to 15 can be configured by any of hardware, Digital Signal Processor (DSP), and software. For example, in the case of being configured by software, each of the above functional blocks 11 to 15 actually include a CPU, a RAM, a ROM, etc., of a computer, and is implemented by operating a program stored in a recording medium such as a RAM, a ROM, a hard disk, or a semiconductor memory.

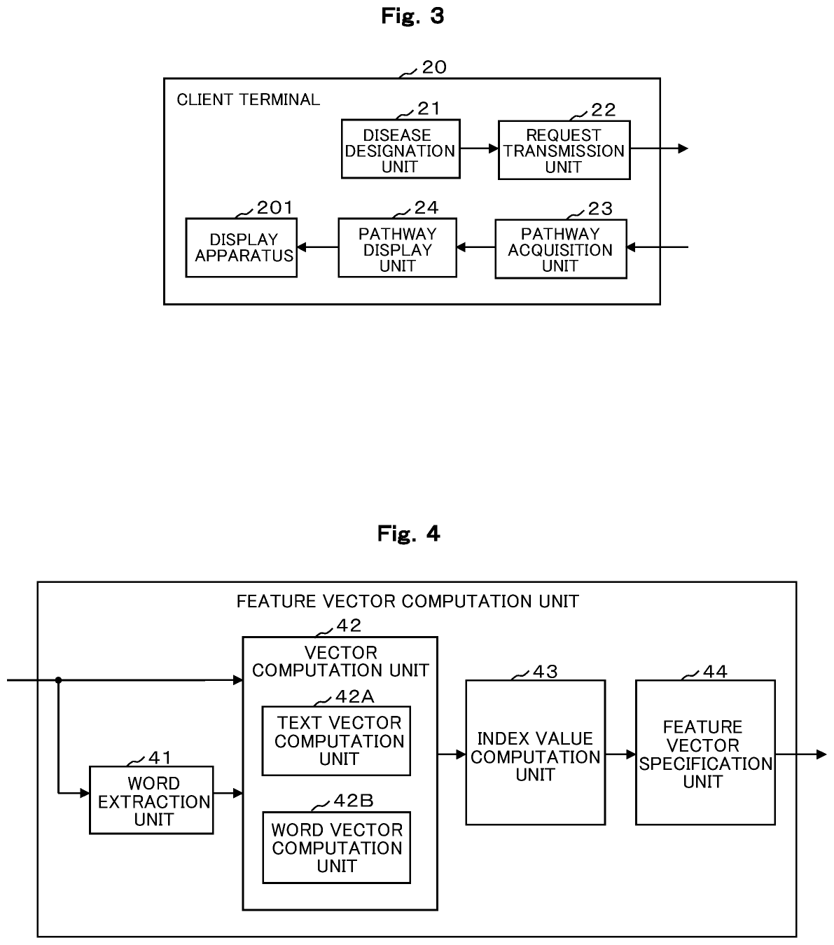

FIG. 3 is a block diagram illustrating a functional configuration example of the client terminal 20 according to the present embodiment. As illustrated in FIG. 3, the client terminal 20 of the present embodiment includes a disease designation unit 21, a request transmission unit 22, a pathway acquisition unit 23, and a pathway display unit 24 as functional configurations. Further, the client terminal 20 of the present embodiment includes a display apparatus 201 such as a liquid crystal display or an organic EL display as hardware.

Each of the above functional blocks 21 to 24 can be configured by any of hardware, DSP, and software. For example, in the case of being configured by software, each of the above functional blocks 21 to 24 actually include a CPU, a RAM, a ROM, etc., of a computer, and is implemented by operating a program stored in a recording medium such as a RAM, a ROM, a hard disk, or a semiconductor memory.

The disease designation unit 21 of the client terminal 20 designates a disease to be analyzed based on a user operation on the client terminal 20. For example, a disease to be analyzed is designated by a user of the client terminal 20 operating a keyboard or a touch panel and inputting a name of the disease to be analyzed. Note that the disease to be analyzed may be designated by the user of the client terminal 20 operating a mouse or the touch panel and selecting the name of the disease to be analyzed from a display list.

The request transmission unit 22 transmits a pathway acquisition request including the disease name designated by the disease designation unit 21 to the server apparatus 10. The pathway acquisition unit 23 acquires pathway data generated by the server apparatus 10 from the server apparatus 10 as a response to the pathway acquisition request transmitted by the request transmission unit 22. The pathway display unit 24 causes the display apparatus 201 to display the pathway based on the pathway data acquired by the pathway acquisition unit 23.

The disease feature vector specification unit 11 of the server apparatus 10 specifies a feature vector (hereinafter referred to as a disease feature vector) corresponding to the disease name (disease name designated as an analysis target by the disease designation unit 21) included in the pathway acquisition request received from the client terminal 20. The disease feature vector is data representing features of the disease (features that can identify the disease) as a combination of values of a plurality of elements. In the present embodiment, as an example, a vector representing a text to which a disease name included as a word in a plurality of texts contributes and a degree at which the disease name contributes to the text is used as a disease feature vector.

The text to be target in the present embodiment may include one sentence (a unit separated by a period) (one statement), or include a plurality of sentences. A text including a plurality of sentences may be a part or all of a text contained in one document. The target text is not limited to a description related to a disease, and may include a description of various other themes.

While a disease name as a word tends to be used in a text describing a disease, the disease name tends not to be used in a text unrelated to the disease. In addition, among texts describing a disease, a text containing a certain disease name as a word is a text describing the disease, and it is highly possible that the disease name is not included in a text describing another type of disease. That is, a text containing a disease name as a word tends to differ depending on the type of disease which is a theme of the text. Therefore, a vector representing a text to which a disease name contributes and a degree at which the disease name contributes to the text may be used as a feature vector that can identify a disease.

For example, the disease feature vector specification unit 11 reads a disease feature vector corresponding to a disease name designated by the disease designation unit 21 from a database (not illustrated) associating and storing the disease name with the disease feature vector corresponding thereto, thereby specifying the disease feature vector. The disease feature vector stored in this database is computed in advance by a feature vector computation apparatus (not illustrated in FIGS. 1 to 3).

As another example, the disease feature vector specification unit 11 may compute a disease feature vector in real time from a disease name included in the pathway acquisition request when a pathway acquisition request is received from the client terminal 20. That is, the disease feature vector specification unit 11 may have a function of the feature vector computation apparatus described above, and the disease feature vector may be specified by executing the function of the feature vector computation apparatus.

FIG. 4 is a block diagram illustrating a functional configuration example of the feature vector computation apparatus. The feature vector computation apparatus illustrated in FIG. 4 inputs text data related to a text, and computes and outputs a disease feature vector reflecting a relationship between the text and a word contained therein. When the disease feature vector specification unit 11 has a function of this feature vector computation apparatus and computes the disease feature vector in real time, the server apparatus 10 stores text data related to a plurality of texts, and the disease feature vector specification unit 11 computes the disease feature vector using the text data.

As illustrated in FIG. 4, the feature vector computation apparatus includes a word extraction unit 41, a vector computation unit 42, an index value computation unit 43, and a feature vector specification unit 44 as functional configurations thereof. The vector computation unit 42 includes a text vector computation unit 42A and a word vector computation unit 42B as more specific functional configurations.

Each of the functional blocks 41 to 44can be configured by any of hardware, a DSP, and software. For example, in the case of being configured by software, each of the functional blocks 41 to 44 actually includes a CPU, a RAM, a ROM, etc. of a computer, and is implemented by operation of a program stored in a recording medium such as a RAM, a ROM, a hard disk, or a semiconductor memory.

The word extraction unit 41 analyzes m texts (m is an arbitrary integer of 2 or more) and extracts n words (n is an arbitrary integer of 2 or more) from the m texts. As a method of analyzing texts, for example, a known morphological analysis can be used. The word extraction unit 41 may extract morphemes of all parts of speech divided by the morphological analysis as words, or may extract only morphemes of a specific part of speech as words.

Note that the same word may be included in the m texts a plurality of times. In this case, the word extraction unit 41 does not extract the plurality of the same words, and extracts only one. That is, the n words extracted by the word extraction unit 41 refer to n types of words.

The vector computation unit 42 computes m text vectors and n word vectors from the m texts and the n words. Here, the text vector computation unit 42A converts each of the m texts to be analyzed by the word extraction unit 41 into a q-dimensional vector (q is an arbitrary integer of 2 or more) according to a predetermined rule, thereby computing the m text vectors including q axis components. In addition, the word vector computation unit 42B converts each of the n words extracted by the word extraction unit 41 into a q-dimensional vector according to a predetermined rule, thereby computing the n word vectors including q axis components.

In the present embodiment, as an example, a text vector and a word vector are computed as follows. Now, a set S=<d∈D, w∈W> including them texts and the n words is considered. Here, a text vector dᵢ→ and a word vector wⱼ→ (hereinafter, the symbol “→” indicates a vector) are associated with each text dᵢ (i=1, 2, . . . , m) and each word wⱼ (j=1, 2, . . . , n), respectively. Then, a probability P(wⱼ|dᵢ) shown in the following Equation (1) is calculated with respect to an arbitrary word wⱼ and an arbitrary text dᵢ.

[Equation 1]

$$P ( {w}_{j} \vert {d}_{i} ) = \frac{exp ( {\vec{w}}_{j} \cdot {\vec{d}}_{i} )}{{\sum }_{k = 1}^{n} exp ( {\vec{w}}_{k} \cdot {\vec{d}}_{i} )} \qquad ( 1 )$$

Note that the probability P(wⱼ|dᵢ) is a value that can be computed in accordance with a probability p disclosed in, a follow known document. “‘Distributed Representations of Sentences and Documents’ by Quoc Le and Tomas Mikolov, Google Inc; Proceedings of the 31st International Conference on Machine Learning Held in Bej ing, China on 22-24 Jun. 2014” This known document states that, for example, when there are three words “the”, “cat”, and “sat”, “on” is predicted as a fourth word, and a computation formula of the prediction probability p is described.

The probability p(wt|wt−k, . . . , wt+k) described in the known document is a correct answer probability when another word wt is predicted from a plurality of words wt−k, . . . , wt+k. Meanwhile, the probability P(wⱼ|dᵢ) shown in Equation (1) used in the present embodiment represents a correct answer probability that one word wⱼ of n words is predicted from one text dᵢ of m texts. Predicting one word wⱼ from one text dᵢ means that, specifically, when a certain text dᵢ appears, a possibility of including the word wⱼ in the text dᵢ is predicted.

Note that since Equation (1) is symmetrical with respect to dᵢ and wⱼ, a probability P(dᵢ|wⱼ) that one text dᵢ of m texts is predicted from one word wⱼ of n words may be calculated. Predicting one text dᵢ from one word wⱼ means that, when a certain word wⱼ appears, a possibility of including the word wⱼ in the text dᵢ is predicted.

In Equation (1), an exponential function value is used, where e is the base and the inner product of the word vector w→ and the text vector d→ is the exponent. Then, a ratio of an exponential function value calculated from a combination of a text dᵢ and a word wⱼ to be predicted to the sum of n exponential function values calculated from each combination of the text dᵢ and n words wₖ (k=1, 2, . . . , n) is calculated as a correct answer probability that one word wⱼ is expected from one text dᵢ.

Here, the inner product value of the word vector wⱼ→ and the text vector dᵢ→ can be regarded as a scalar value when the word vector wⱼ→ is projected in a direction of the text vector dᵢ→, that is, a component value in the direction of the text vector dᵢ→ included in the word vector wⱼ→, which can be considered to represent a degree at which the word wⱼ contributes to the text dᵢ. Therefore, obtaining the ratio of the exponential function value calculated for one word Wⱼ to the sum of the exponential function values calculated for n words wₖ (k=1, 2, . . . , n) using the exponential function value calculated using the inner product corresponds to obtaining the correct answer probability that one word wⱼ of n words is predicted from one text dᵢ.

Note that here, a calculation example using the exponential function value using the inner product value of the word vector w→ and the text vector d→ as an exponent has been described. However, the exponential function value may not be used. Any calculation formula using the inner product value of the word vector w→ and the text vector d→ may be used. For example, the probability may be obtained from the ratio of the inner product values itself.

Next, the vector computation unit 42 computes the text vector dᵢ→ and the word vector wⱼ∝3 that maximize a value L of the sum of the probability P (wⱼ|dᵢ) computed by Equation (1) for all the set S as shown in the following Equation (2). That is, the text vector computation unit 42A and the word vector computation unit 42B compute the probability P (wⱼ|dᵢ) computed by Equation (1) for all combinations of the m texts and the n words, and compute the text vector dᵢ→ and the word vector wⱼ→ that maximize a target variable L using the sum thereof as the target variable L.

$$\begin{matrix}[ Equation 2 ] & \\ L = {\sum }_{d ∈ D} {\sum }_{w ∈ W} \# ( w , d ) p ( w \vert d ) & ( 2 )\end{matrix}$$

Maximizing the total value L of the probability P (wⱼ|dᵢ) computed for all the combinations of the m texts and the n words corresponds to maximizing the correct answer probability that a certain word wⱼ (j=1, 2, . . . , n) is predicted from a certain text dᵢ (i=1, 2, . . . , m). That is, the vector computation unit 42 can be considered to compute the text vector dᵢ→ and the word vector wⱼ→ that maximize the correct answer probability.

Here, in the present embodiment, as described above, the vector computation unit 42 converts each of the m texts dᵢ into a q-dimensional vector to compute the m texts vectors dᵢ→ including the q axis components, and converts each of the n words into a q-dimensional vector to compute the n word vectors wⱼ→ including the q axis components, which corresponds to computing the text vector dᵢ→ and the word vector wⱼ→ that maximize the target variable L by making q axis directions variable.

The index value computation unit 43 takes each of the inner products of the m text vectors dᵢ→ and the n word vectors wⱼ→ computed by the vector computation unit 42, thereby computing index values reflecting the relationship between the m texts di and the n words wⱼ. In the present embodiment, as shown in the following Equation (3), the index value computation unit 43 obtains the product of a text matrix D having the respective q axis components (d₁₁ to d_{mq}) of the m text vectors dᵢ→ as respective elements and a word matrix W having the respective q axis components (w₁₁ to w_{nq}) of the n word vectors wⱼ→ as respective elements, thereby computing an index value matrix DW having m×n index values as elements. Here, W^{t} is the transposed matrix of the word matrix.

$$\begin{matrix}[ Equation 3 ] & \\ D = ( \begin{matrix}{d}_{11} & {d}_{12} & … & {d}_{1 q} \\ {d}_{21} & {d}_{22} & … & {d}_{2 q} \\ ⋮ & ⋮ & ⋱ & ⋮ \\ {d}_{m 1} & {d}_{m 2} & … & {d}_{mq}\end{matrix} ) W = ( \begin{matrix}{w}_{11} & {w}_{12} & … & {w}_{1 q} \\ {w}_{21} & {w}_{22} & … & {w}_{2 q} \\ ⋮ & ⋮ & ⋱ & ⋮ \\ {w}_{m 1} & {w}_{m 2} & … & {w}_{mq}\end{matrix} ) & ( 3 )\end{matrix}$$

$$DW = D * {W}^{t} = ( \begin{matrix}{dw}_{11} & {dw}_{12} & … & {dw}_{1 n} \\ {dw}_{21} & {dw}_{22} & … & {dw}_{2 n} \\ ⋮ & ⋮ & ⋱ & ⋮ \\ {dw}_{m 1} & {dw}_{m 2} & … & {dw}_{mn}\end{matrix} )$$

Each element of the index value matrix DW computed in this manner may indicate which word contributes to which text and to what extent and which text contributes to which word and to what extent. For example, an element dw₁₂ in the first row and the second column may be a value indicating a degree at which the word w₂ contributes to a text d₁ and may be a value indicating a degree at which the text di contributes to a word w₂. In this way, each row of the index value matrix DW can be used to evaluate the similarity of a text, and each column can be used to evaluate the similarity of a word.

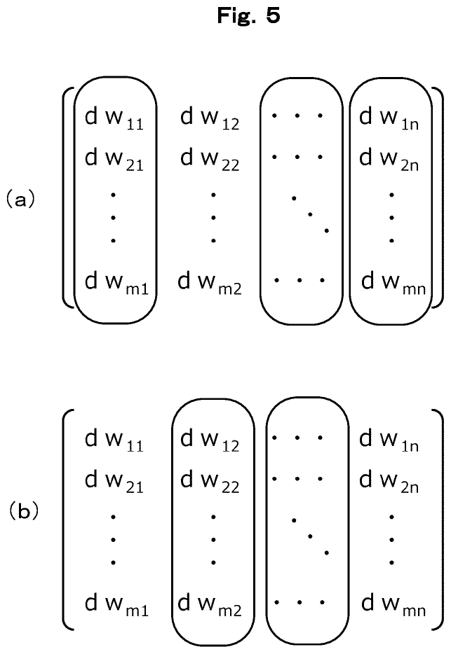

The feature vector specification unit 44 specifies, as a disease feature vector, a word index value group including m index values for one disease name for each of a plurality of disease names among n words. That is, as illustrated in FIG. 5(a), the feature vector specification unit 44 specifies, as a disease feature vector corresponding to each disease name, a word index value group related to a word corresponding to a disease name among n sets of word index value groups (m index values per column) constituting respective columns of the index value matrix DW.

Returning to FIG. 2, a configuration of the server apparatus 10 will be described. The related molecule estimation unit 12 inputs a disease feature vector specified by the disease feature vector specification unit 11 for a disease to be analyzed to a first trained model stored in advance in the first model storage unit 101, thereby estimating a plurality of molecules associated with the disease. Here, the first trained model is subjected to machine learning so as to output information about a molecule corresponding to the molecule feature vector similar to the disease feature vector when the disease feature vector is input based on a similarity between the disease feature vector and the molecule feature vector.

A form of the first trained model stored in the first model storage unit 101 may be any of a regression model, a tree model, a neural network model, a Bayesian model, a clustering model, etc. Note that the models listed here are merely examples, and the first trained model is not limited thereto. For example, it is possible to adopt a function model that computes a similarity between a disease feature vector and a molecule feature vector and outputs information about a molecule corresponding to the molecule feature vector whose similarity to the disease feature vector is equal to or more than a predetermined value.

The molecule feature vector used here is data representing a feature (feature that can identify a molecule) of a molecule of a protein, a gene, etc. as a combination of values of a plurality of elements. In the present embodiment, as an example, a vector representing a text to which a molecule name included as a word in a plurality of texts contributes and a degree at which the molecule name contributes to the text is used as a molecule feature vector. This molecule feature vector can be computed by the feature vector computation apparatus illustrated in FIG. 4.

That is, the feature vector specification unit 44 specifies, as a molecule feature vector, a word index value group including m index values for one molecule name for each of a plurality of molecule names among n words. Specifically, as illustrated in FIG. 5(b), the feature vector specification unit 44 specifies, as a molecule feature vector corresponding to each molecule name, a word index value group related to a word corresponding to a molecule name among n sets of word index value groups (m index values per column) constituting respective columns of the index value matrix DW.

The feature vector computation apparatus described above computes a disease feature vector related to a plurality of disease names and computes a molecule feature vector related to a plurality of molecule names. Then, machine learning of the first trained model is performed in advance using these data sets, and the first trained model learned based on the similarity between the disease feature vector and the molecule feature vector is stored in the first model storage unit 101.

Here, the similarity between the disease feature vector and the molecule feature vector can be evaluated by various methods. For example, it is possible to apply a method of extracting a feature quantity using a predetermined function for each of the disease feature vector and the molecule feature vector and evaluating a similarity of the feature quantity. Alternatively, it is possible to use a Euclidean distance or cosine similarity between the word index value group of the disease feature vector and the word index value group of the molecule feature vector, or it is possible to use an edit distance.

The fact that the disease feature vector and the molecule feature vector are similar to each other means that a property indicating a text to which a word as a disease name contributes and a degree at which the word contributes to the text is similar to a property indicating a text to which a word as a molecule name contributes and a degree at which the word contributes to the text. Since the text is described according to a specific theme, the disease name and the molecule name, which have a similar relationship between the disease feature vector and the molecule feature vector, mean that contributions to a plurality of texts described in relation to each theme are similar, and it is possible to presume that there is some association between the disease and the molecule.

In case that the disease name and the molecule name are described in one text, it is clear that the disease and the molecule are related. On the other hand, when the disease name and the molecule name are described across a plurality of texts, it is unclear whether there is relevance between a disease described in one text and a molecule described in another text, and even when medical personnel read these texts, it is difficult to immediately understand that there is relevance.

On the other hand, according to the present embodiment, even when a disease name and a molecule name are described across a plurality of texts in this way, it is possible to presume that there may be some relevance between the disease and the molecule. In this way, when a disease feature vector corresponding to a certain disease name is input to the first trained model, even a molecule whose relevance to the disease is unknown may be output as a related molecule by estimation based on learning.

The molecular property estimation unit 13 inputs a disease feature vector specified by the disease feature vector specification unit 11 for a disease to be analyzed and a molecule feature vector specified for a plurality of molecules estimated by the related molecule estimation unit 12 to the second trained model stored in the second model storage unit 102, thereby estimating a probability that a molecule acting on the disease is causative or responsive as a property for each of a plurality of molecules presumed to be associated with the disease.

A form of the second trained model stored in the second model storage unit 102 may be any of a regression model, a tree model, a neural network model, a Bayesian model, a clustering model, etc. Note that the models listed here are merely examples, and the second trained model is not limited thereto.

Here, the second trained model is subjected to machine learning so as to output a probability that a property of a molecule is causative or responsive when a disease feature vector and a molecule feature vector are input using the disease feature vector, the molecule feature vector, and a data set of property information representing the property of the molecule acting on a disease as teacher data. The causativeness is a property that may cause a disease due to the presence or mutation of the molecule. Responsiveness is a property that a molecule may mutate due to the onset of a disease. In the present embodiment, as an example, the second trained model will be described as outputting a probability that a property of a molecule with respect to a disease is causative.

With regard to a known disease, there is known information about which molecule is causative and which molecule is responsive. The second trained model is created by setting a disease feature vector, a molecule feature vector, and a data set of property information of a molecule generated from this known information as teacher data (property information of a molecule is set as correct answer data) and performing machine learning using this data set. Therefore, for a molecule whose property is known to be causative for a known disease, a high probability value is output from the second trained model. On the other hand, for a molecule whose property is known to be responsive for a known disease, a low probability value is output from the second trained model.

In addition, a molecule whose relevance to a disease is unknown in human knowledge so far may be included in a plurality of molecules whose relevance to the disease is estimated by the related molecule estimation unit 12. Even for such a molecule, by estimation based on learning, a value of a probability indicating that that molecule may exhibit a causative property with respect to the disease is output from the second trained model.

That is, in case that a similarly between (a feature quantity obtained from) a combination of a disease feature vector corresponding to a certain disease and a molecule feature vector corresponding to a molecule whose relevance to the disease is unknown and (a feature quantity obtained from) a combination of a disease feature vector corresponding to the certain disease and a molecule feature vector corresponding to a molecule known to be causative is high, a relatively high probability value tends to be output from the second trained model.

On the other hand, in case that a similarly between (a feature quantity obtained from) a combination of a disease feature vector corresponding to a certain disease and a molecule feature vector corresponding to a molecule whose relevance to the disease is unknown and (a feature quantity obtained from) a combination of a disease feature vector corresponding to the certain disease and a molecule feature vector corresponding to a molecule known to be responsive is high, a relatively low probability value tends to be output from the second trained model.

As described above, the molecular property estimation unit 13 estimates the property of the action of the molecule on the disease by inputting the disease feature vector and the molecule feature vector to the second trained model. Here, as the disease feature vector, one specified by the disease feature vector specification unit 11 is used. On the other hand, as the molecule feature vector, a plurality of molecules estimated by the related molecule estimation unit 12, that is, a molecule feature vector corresponding to a molecule information list output from the first trained model is used.

With regard to the molecule feature vector, for example, the molecular property estimation unit 13 reads a molecule feature vector corresponding to a molecule name estimated by the related molecule estimation unit 12 from a database (not illustrated) that associates and stores a molecule name with a molecule feature vector corresponding thereto. Note that the molecular property estimation unit 13 may compute the molecule feature vector from molecular names thereof in real time when a molecule information list is output from the first trained model. That is, the molecular property estimation unit 13 may have a function of the feature vector computation apparatus described above and specify a molecule feature vector by executing the function of the feature vector computation apparatus.

As another example, as the first trained model used in the related molecule estimation unit 12, it is possible to use one subjected to machine learning so as to output a molecule feature vector similar to a disease feature vector when the disease feature vector is input based on a similarity between the disease feature vector and the molecule feature vector. In this case, the molecular property estimation unit 13 can input the disease feature vector output from the disease feature vector specification unit 11 and the molecule feature vector output from the related molecule estimation unit 12 to the second trained model directly.

The pathway generation unit 14 generates a pathway representing an intermolecular interaction as a route map in a manner that a causative molecule is on an upstream side and a responsive molecule is on an downstream side and that an intermolecular connection relationship shown by a known knowledge database is reflected for a plurality of molecules whose relevance to the disease is estimated by the related molecule estimation unit 12 by using a property of a molecule estimated by the molecular property estimation unit 13 and the knowledge database showing the intermolecular connection relationship.

In this instance, for example, the pathway generation unit 14 generates the pathway in a manner that a molecule (hereinafter referred to as a causative molecule) whose probability value estimated to be causative by the molecular property estimation unit 13 is larger than a first threshold Th1 is disposed on the upstream side of the pathway, a molecule (hereinafter referred to as a responsive molecule) whose probability value is smaller than a second threshold Th2 (Th1>Th2) is disposed on the downstream side of the pathway, and a molecule (hereinafter referred to as a linking molecule) whose probability value is larger than or equal to the second threshold Th2 and smaller than or equal to the first threshold Th1 is disposed between the causative molecule and the responsive molecule.

The known knowledge database showing the intermolecular connection relationship is stored in advance in the knowledge DB storage unit 103. For example, the intermolecular connection relationship includes a relationship in which when an expression level of a certain molecule increases (or decreases), an expression level of another molecule increases (decreases) in conjunction with the increase (decrease). The knowledge DB storage unit 103 stores in advance known information about such an intermolecular relationship. However, the known information contained in the knowledge database stored in the knowledge DB storage unit 103 is limited to information indicating which molecule has a relationship with which molecule, and does not include information indicating the magnitude of the connection relationship. In addition, the known information is a set of information showing a connection relationship between two molecules, and is not information showing a sequential connection relationship between three or more molecules.

On the other hand, for example, the pathway generation unit 14 supplements the magnitude of the intermolecular relationship not contained in the known information by useing a value of a degree of similarity between the disease feature vector and the molecule feature vector specified by the related molecule estimation unit 12. For example, it is possible to generate a pathway by facilitating the connection between these molecules on the assumption that the molecules having similar similarity values have a strong relationship with each other. In addition, for example, the pathway generation unit 14 uses a minimum flow algorithm to specify a sequential connection relationship between three or more molecules.

As described above, the pathway generation unit 14 generates a pathway that represents an interaction between three or more molecules as a route map by setting the causative molecule on the upstream side and the responsive molecule on the downstream side, and reflecting the connection relationship shown by the knowledge database. Note that even though an example of using the minimum flow algorithm has been described here, the invention is not limited thereto.

The pathway provision unit 15 provides the client terminal 20 with the pathway data generated by the pathway generation unit 14. As described above, in the client terminal 20, the pathway acquisition unit 23 acquires the pathway data provided from the server apparatus 10, and the pathway display unit 24 causes the display apparatus 201 to display the pathway.

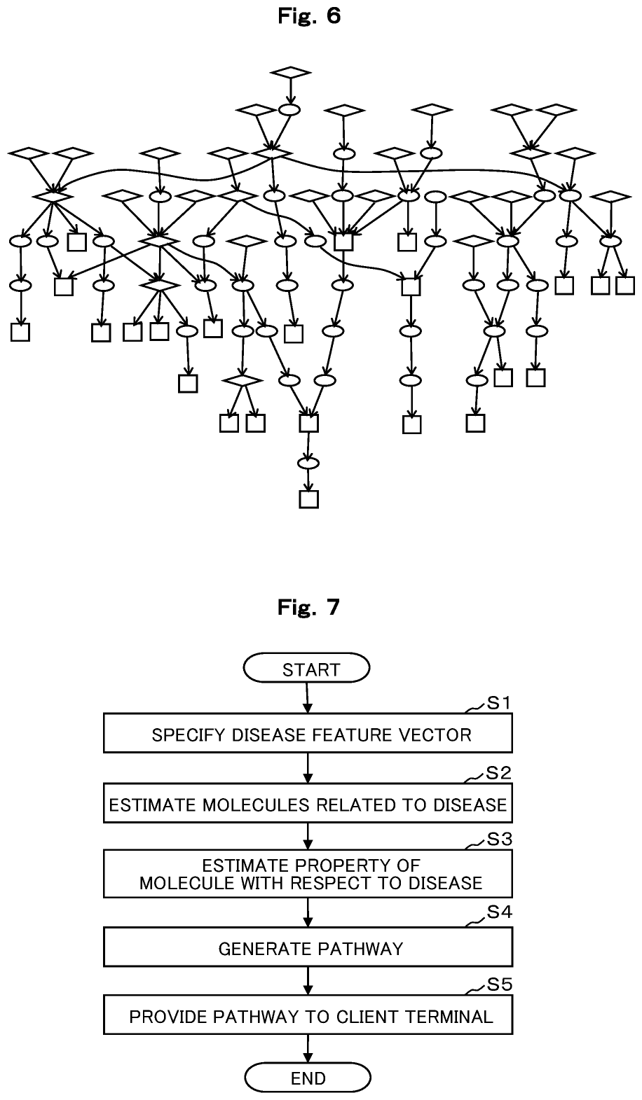

FIG. 6 is a diagram illustrating an example of a pathway displayed on the display apparatus 201. In FIG. 6, a diamond-shaped symbol mainly shown on the upstream side of the pathway is the causative molecule, a square symbol mainly shown on the downstream side is the responsive molecule, and an elliptical symbol is the linking molecule. Even though a molecule name is not written on each symbol for convenience of drawing, the molecule name is actually displayed on each symbol.

As mentioned above, this pathway may include a molecule whose relevance to a disease is unknown, and may include connectivity (intermolecular interaction) in which a property of a molecule with respect to the disease is unknown. By viewing such a pathway, for a disease to be analyzed, the user of the client terminal 20 can easily detect a certain molecule that may be related to the disease or a certain molecule that may be affected when a molecule is operated and the molecule that affects the certain molecule.

FIG. 7 is a flowchart illustrating an operation example of the server apparatus 10 (pathway generation apparatus) according to the present embodiment configured as described above. The flowchart illustrated in FIG. 7 starts when the disease feature vector specification unit 11 receives a pathway acquisition request from the client terminal 20.

The disease feature vector specification unit 11 specifies a disease feature vector corresponding to a disease name contained in a pathway acquisition request (step S1). Subsequently, the related molecule estimation unit 12 inputs the disease feature vector specified by the disease feature vector specification unit 11 to a first trained model, thereby estimating a plurality of molecules related to the disease (step S2). That is, the related molecule estimation unit 12 inputs the disease feature vector to the first trained model, and outputs an information list of a molecule corresponding to a molecule feature vector similar to the disease feature vector from the first trained model.

Subsequently, the molecular property estimation unit 13 inputs the disease feature vector specified by the disease feature vector specification unit 11 and a plurality of molecule feature vectors specified for a plurality of molecules estimated by the related molecule estimation unit 12 to a second trained model, thereby estimating a probability that a property of a molecule acting on the disease is causative for each of a plurality of molecules presumed to be associated with the disease (step S3).

Subsequently, the pathway generation unit 14 generates a pathway representing an intermolecular interaction as a route map in a manner that a causative molecule is on an upstream side and a responsive molecule is on an downstream side and that a connection relationship shown by a knowledge database is reflected for a plurality of molecules whose relevance to the disease is estimated by the related molecule estimation unit 12 by using a property of a molecule estimated by the molecular property estimation unit 13 and the knowledge database stored in the knowledge DB storage unit 103 (step S4).

Then, the pathway provision unit 15 provides data of the pathway generated by the pathway generation unit 14 to the client terminal 20 which is a transmission source of the pathway acquisition request (step S5) . As a result, processing of the flowchart illustrated in FIG. 7 is completed.

As described in detail above, according to the present embodiment, when a disease feature vector for a disease to be analyzed is input to the first trained model, not only a molecule known to be related to the disease, but also a molecule whose relevance to the disease is unknown may be output as a related molecule by estimation based on learning. In addition, when a molecule feature vector of the molecule estimated in this way and the disease feature vector are input to the second trained model, a probability indicating whether a molecule may exhibit a causative or responsive property with respect to the disease is output not only for the molecule whose relevance to the disease is known but also the molecule whose relevance is unknown by estimation based on learning. Then, an estimation result with regard to a property of a molecule presumed to be associated with the disease in this way and the known knowledge database showing the intermolecular connection relationship are used to generate a pathway representing an intermolecular interaction as a route map.

As described above, according to the pathway generation apparatus of the present embodiment, it is possible to generate a pathway useful for obtaining new knowledge beyond a range of a known intermolecular interaction described in a literature, etc. , and the pathway can be effectively used for research and development of treatment, drug discovery, etc. of a disease. For example, an important molecule related to a new disease can be visually comprehended by the pathway, and it is possible to discover concomitant drug candidates for efficiently blocking a plurality of paths and to predict efficacy and safety of drug administration while being able to discover a possibility of treatment using an existing drug and a new target or biomarker which is inconceivable in existing knowledge based on this visualized information.

Note that in the above embodiment, a description has been given of an example in which the first trained model and the second trained model are created in advance and stored in the first model storage unit 101 and the second model storage unit 102. The apparatus that performs machine learning may be configured as an apparatus different from the server apparatus 10, or the server apparatus 10 may be configured to have a function of performing machine learning.

Further, in the embodiment, a description has been given of an example in which the feature vector computed by the feature vector computation apparatus illustrated in FIG. 4 is used as the disease feature vector and the molecule feature vector. However, the invention is not limited thereto. For example, in case that a vector represents a text to which a disease name or a molecule name contained as a word in a plurality of texts contributes and a degree at which the disease name or the molecule name contributes to the text, the vector is not limited to the feature vector computed by the feature vector computation apparatus illustrated in FIG. 4. In addition, the feature vector may not be obtained from a relationship between a text and a word. When the feature of the disease or the molecule can be identified, the vector may be used as the disease feature vector or the molecule feature vector.

When the feature vector computed by the feature vector computation apparatus illustrated in FIG. 4 is used as the disease feature vector and the molecule feature vector, there are advantages that the disease feature vector and the molecule feature vector can be extracted from one index value matrix DW computed by one algorithm, and mutual similarity or relationship can be more logically specified. Thus, it is possible to improve certainty of an estimation result performed by using the first trained model and the second trained model, and to enhance usefulness of a generated pathway.

Further, in the embodiment, it has been described that for a text to be targeted when a disease feature vector and a molecule feature vector are created, without being limited to a description of a disease, a description of various other themes maybe included. However, the invention is not limited thereto. For example, only a text containing description content related to a specific disease may be targeted. In this case, by utilizing a property that a word index value group (n index values per row) constituting each row of the index value matrix DW can be used as index value in order to evaluate a similarly of a text, the word index value group of each row may be used as a text feature vector to extract a plurality of texts whose text feature vectors are similar to each other.

In addition, all the embodiments are merely examples of embodiment in carrying out the invention, and the technical scope of the invention should not be construed in a limited manner by the embodiments. That is, the invention can be implemented in various forms without departing from a gist or a main feature thereof.

### REFERENCE SIGNS LIST

10 Server apparatus (pathway generation apparatus)

11 Disease feature vector specification unit

12 Related molecule estimation unit

13 Molecular property estimation unit

14 Pathway generation unit

101 First model storage unit

102 Second model storage unit

103 Knowledge DB storage unit

1. A pathway generation apparatus characterized by comprising:
a related molecule estimation unit that inputs a disease feature vector specified for a disease to be analyzed to a first trained model, thereby estimating a plurality of molecules related to the disease;
a molecular property estimation unit that inputs a disease feature vector specified for the disease to be analyzed and a molecule feature vector specified for the plurality of molecules estimated by the related molecule estimation unit to a second trained model, thereby estimating a probability that a molecule is causative or responsive as a property acting on the disease for each of the plurality of molecules; and
a pathway generation unit that generates a pathway representing an intermolecular interaction as a route map in a manner that a causative molecule is on an upstream side and a responsive molecule is on an downstream side and that an intermolecular connection relationship shown by a known knowledge database is reflected for the plurality of molecules estimated by the related molecule estimation unit by using a property of a molecule estimated by the molecular property estimation unit and the knowledge database showing the intermolecular connection relationship.

2. The pathway generation apparatus according to claim 1, characterized in that the first trained model is subjected to machine learning so as to output information about a molecule feature vector similar to the disease feature vector or a molecule corresponding to the molecule feature vector when the disease feature vector is input based on a similarity between the disease feature vector and the molecule feature vector.

3. The pathway generation apparatus according to claim 1, characterized in that the second trained model is subjected to machine learning so as to output a probability that a property of a molecule is causative or responsive when the disease feature vector and the molecule feature vector are input using the disease feature vector, the molecule feature vector, and a data set of property information representing the property of the molecule acting on the disease as teacher data.

4. The pathway generation apparatus according to claim 1,
characterized in that the disease feature vector is a vector representing a text to which a disease name contained as a word in a plurality of texts contributes and a degree at which the disease name contributes to the text, and
the molecule feature vector is a vector representing a text to which a molecule name contained as a word in a plurality of texts contributes and a degree at which the molecule name contributes to the text.

5. The pathway generation apparatus according to claim 4,
characterized in that the disease feature vector and the molecule feature vector are computed by
a word extraction process of analyzing m texts (m is an arbitrary integer of 2 or more), and extracts n words (n is an arbitrary integer of 2 or more) from the m texts;
a text vector computation process of converting each of the m texts into a q-dimensional vector (q is an arbitrary integer of 2 or more) according to a predetermined rule, thereby computing m text vectors including q axis components;
a word vector computation process of converting each of the n words into a q-dimensional vector according to a predetermined rule, thereby computing n word vectors including q axis components;
an index value computation process of obtaining each of inner products of the m text vectors and the n word vectors, thereby computing m x n index values reflecting a relationship between the m texts and the n words;
a disease feature vector specification process of specifying, as the disease feature vector, a word index value group including m index values for one disease name with respect to disease names contained in the n words; and
a molecule feature vector specification process of specifying, as the molecule feature vector, a word index value group including m index values for one molecule name with respect to molecule names contained in the n words.

6. The pathway generation apparatus according to claim 4, characterized by further comprising:
a word extraction unit that analyzes m texts (m is an arbitrary integer of 2 or more), and extracts n words (n is an arbitrary integer of 2 or more) from the m texts;
a text vector computation unit that converts each of the m texts into a q-dimensional vector (q is an arbitrary integer of 2 or more) according to a predetermined rule, thereby computing m text vectors including q axis components;
a word vector computation unit that converts each of the n words into a q-dimensional vector according to a predetermined rule, thereby computing n word vectors including q axis components;
an index value computation unit that obtains each of inner products of the m text vectors and the n word vectors, thereby computing m x n index values reflecting a relationship between the m texts and the n words; and
a feature vector specification unit that specifies, as the disease feature vector or the molecule feature vector, a word index value group including m index values for one disease name or molecule name with respect to disease names or molecule names contained in the n words.

7. A pathway generation method, the method being characterized by comprising:
a first step of inputting a disease feature vector specified for a disease to be analyzed to a first trained model, thereby estimating a plurality of molecules related to the disease by a related molecule estimation unit of a computer;
a second step of inputting a disease feature vector specified for the disease to be analyzed and a molecule feature vector specified for the plurality of molecules estimated by the related molecule estimation unit to a second trained model, thereby estimating a probability that a molecule is causative or responsive as a property acting on the disease for each of the plurality of molecules by a molecular property estimation unit of the computer; and
a third step of generating a pathway representing an intermolecular interaction as a route map in a manner that a causative molecule is on an upstream side and a responsive molecule is on an downstream side and that an intermolecular connection relationship shown by a known knowledge database is reflected for the plurality of molecules estimated by the related molecule estimation unit by using a property of a molecule estimated by the molecular property estimation unit and the knowledge database showing the intermolecular connection relationship by a pathway generation unit of the computer.

8. A pathway generation program stored on a non-transitory computer readable medium, the program for causing the computer to function as:
a related molecule estimation means that inputs a disease feature vector specified for a disease to be analyzed to a first trained model, thereby estimating a plurality of molecules related to the disease;
a molecular property estimation means that inputs a disease feature vector specified for the disease to be analyzed and a molecule feature vector specified for the plurality of molecules estimated by the related molecule estimation means to a second trained model, thereby estimating a probability that a molecule is causative or responsive as a property acting on the disease for each of the plurality of molecules; and
a pathway generation means that generates a pathway representing an intermolecular interaction as a route map in a manner that a causative molecule is on an upstream side and a responsive molecule is on an downstream side and that an intermolecular connection relationship shown by a known knowledge database is reflected for the plurality of molecules estimated by the related molecule estimation means by using a property of a molecule estimated by the molecular property estimation means and the knowledge database showing the intermolecular connection relationship.

9. The pathway generation apparatus according to claim 2, characterized in that the second trained model is subjected to machine learning so as to output a probability that a property of a molecule is causative or responsive when the disease feature vector and the molecule feature vector are input using the disease feature vector, the molecule feature vector, and a data set of property information representing the property of the molecule acting on the disease as teacher data.

10. The pathway generation apparatus according to claim 2,
characterized in that the disease feature vector is a vector representing a text to which a disease name contained as a word in a plurality of texts contributes and a degree at which the disease name contributes to the text, and
the molecule feature vector is a vector representing a text to which a molecule name contained as a word in a plurality of texts contributes and a degree at which the molecule name contributes to the text.

11. The pathway generation apparatus according to claim 3,
characterized in that the disease feature vector is a vector representing a text to which a disease name contained as a word in a plurality of texts contributes and a degree at which the disease name contributes to the text, and
the molecule feature vector is a vector representing a text to which a molecule name contained as a word in a plurality of texts contributes and a degree at which the molecule name contributes to the text.
